(19) United States
(12) Patent Application Publication
(10) Pub. No.: US 2016/0206358 A1
(43) Pub. Date: Jul. 21, 2016

(54) Orthopedic Fixation System and Method

(71) Applicants: Chunfeng Zhao, Rochester, MN (US); Kai-Nan An, Rochester, MN (US); Andrew R. Thoreson, Kenyon, MN (US)

(72) Inventors: Chunfeng Zhao, Rochester, MN (US); Kai-Nan An, Rochester, MN (US); Andrew R. Thoreson, Kenyon, MN (US)

(21) Appl. No.: 15/003,116

(22) Filed: Jan. 21, 2016

Related U.S. Application Data
(60) Provisional application No. 62/106,015, filed on Jan. 21, 2015.

(51) Int. Cl. A61B 17/86; A61B 17/70

(52) U.S. Cl. CPC A61B 17/8695; A61B 17/707

(57) ABSTRACT

An orthopedic fixation system includes a pedicle screw washer. The pedicle screw washer includes a body, an aperture for receiving a pedicle screw, a first securing member extending from the body spaced from the aperture and dimensioned to engage a first side of a transverse process of a vertebra, and a second securing member extending from the body spaced from the aperture and dimensioned apart from the first securing member to engage a second opposed side of the transverse process of the vertebra.

### CROSS-REFERENCES TO RELATED APPLICATIONS

This application claims priority to U.S. Patent Application No. 62/106,015 filed Jan. 21, 2015.

### STATEMENT REGARDING FEDERALLY SPONSORED RESEARCH

Not Applicable.

### BACKGROUND OF THE INVENTION

1. Field of the Invention

This invention relates generally to orthopedic fixation systems for securing implements used in orthopedic surgery and, more specifically, to securing pedicle screws used in spinal instrumentation surgeries.

2. Description of the Related Art

Spinal instrumentation surgeries can often include the use of one or more pedicle screws. The pedicle screws are typically cancellous bone screws and can sustain high loads, which may render them susceptible to failure by loosening or pullout. Various devices and methods have been developed for locking or fixating pedicle screws; however, most of these devices and methods include complicated coupling elements that are often made up of multiple parts. The multitude of parts can increase the complexity of and time required for the spinal surgeries.

Therefore, it would be desirable to have a system that increases the structural stability of a pedicle screw without increasing the complexity, or the time, required of a surgical procedure used to install the pedicle screw.

### SUMMARY OF THE INVENTION

The aforementioned needs are met by providing an orthopedic fixation system of the present disclosure. The orthopedic fixation system includes a pedicle screw washer. The pedicle screw washer includes a body, an aperture for receiving a pedicle screw, a first securing member extending from the body spaced from the aperture and dimensioned to engage a first side of a transverse process of a vertebra, and a second securing member extending from the body spaced from the aperture and dimensioned apart from the first securing member to engage a second opposed side of the transverse process of the vertebra.

In one aspect, the invention may provide a pedicle screw washer comprising a body and an aperture in the body for receiving a pedicle screw. A first securing member may extend from the body and be dimensioned to engage a first side of a transverse process of a vertebra. Likewise, a second securing member may extend from the body and be dimensioned to engage a second side of the transverse process of the vertebra. In one non-limiting example form, the first securing member and the second securing member each define a substantially rectangular shape.

The pedicle screw washer may further include one or more bone penetrating members extending from the body. In one form, one or more of the bone penetrating members may define a substantially triangular shape. One or more the bone penetrating members may extend from the body in a same direction as at least one of the first and second securing members. The body of the pedicle screw washer may include a first section including one or more bone penetrating members, and a second section including the first and second securing members. One or more bone penetrating members may extend from a periphery of the body. The first section of the body may include a substantially semi-circular peripheral edge. The bone penetrating members may extend from the body at equally spaced intervals along the periphery of the body.

In one non-limiting example form of the pedicle screw washer, the body is fabricated from a generally malleable material such that the first section may be angled with respect to the second section of the body. A first proximal face of the first section of the body may form an obtuse angle with respect to a second proximal face of the second section of the body when installed on the vertebra.

The first securing member may be bendable around the first side of the transverse process, and the second securing member may be bendable around the second opposite side of the transverse process. Before or after bending, the first securing member may include a first distal end section that is inwardly directed toward the second securing member, and the second securing member may include a second distal end section that is inwardly directed toward the first securing member.

In one non-limiting example form of the pedicle screw washer, the aperture in the body defines a substantially rectangular shape. The aperture may include a internal contour that mates and engages with an external contour located on a section of the pedicle screw. The aperture may define a substantially round shape. The aperture may include internal detents for engaging external protrusions of the pedicle screw. The aperture may include internal threads for engaging a threaded section of the pedicle screw.

In another aspect, the invention may provide an orthopedic fixation system comprising a pedicle screw and a pedicle screw washer of the invention. The pedicle screw washer may include a body and an aperture for receiving the pedicle screw. A first securing member may extend from the body and be dimensioned to engage a first side of a transverse process of a vertebra. Likewise, a second securing member may extend from the body and be dimensioned to engage a second side of the transverse process of the vertebra. The pedicle screw washer of the orthopedic fixation system may include one or more of any of the features of the pedicle screw washer of the invention described above.

In one non-limiting example form of the orthopedic fixation system, the pedicle screw includes an externally threaded section and a threaded extension. The aperture in the body of the pedicle screw washer includes internal threads for engaging the externally threaded section of the pedicle screw. The orthopedic fixation system may further comprise a fastening element configured to engage the threaded extension of the pedicle screw and fasten the pedicle screw washer to the vertebra.

In yet another aspect, the invention may provide a method for securing a pedicle screw. The method may comprise placing the pedicle screw washer on a vertebra such that a first securing member, extending from a body of the pedicle screw washer, engages a first side of a transverse process of the vertebra, and such that a second securing member, extending from the body of the pedicle screw washer, engages a second side of the transverse process. The method may further comprise installing the pedicle screw in the vertebra and through an aperture of the pedicle screw washer. The aperture may include internal threads for engaging an externally threaded section of the pedicle screw. The method may include the step of fastening the pedicle screw washer to the vertebra by installing a fastening element on a threaded extension of the pedicle screw. The method may include the step of bending the first and second securing members around and along the first and second sides of the transverse process of the vertebra. The method may include the step of bending the body of the pedicle screw washer along a contour of the vertebra. Bending the body of the pedicle screw washer may form an obtuse angle between a first proximal face of a first section of the body and a second proximal face of a second section of the body, wherein the first section includes the aperture and the second section includes the first and second securing members of the pedicle screw washer. The method may include the step of forcing a bone penetrating member, extending from the body of the pedicle screw washer, into the vertebra.

In still another aspect, the invention may provide a method for securing a pedicle screw. The method may comprise installing the pedicle screw in a vertebra and placing a pedicle screw washer on the vertebra such that an aperture in a body of the pedicle screw washer receives the pedicle screw. The pedicle screw washer may further include a first securing member, extending from the body of the pedicle screw washer, engaging a first side of a transverse process of the vertebra, and a second securing member, extending from the body of the pedicle screw washer, engaging a second side of the transverse process. The method may include the step of bending the first and second securing members around and along the first and second sides of the transverse process of the vertebra. The method may include the step of bending the body of the pedicle screw washer along a contour of the vertebra. Bending the body of the pedicle screw washer may form an obtuse angle between a first proximal face of a first section of the body and a second proximal face of a second section of the body, wherein the first section includes the aperture and the second section includes the first and second securing members of the pedicle screw washer. The method may include the step of forcing a bone penetrating member, extending from the body of the pedicle screw washer, into the vertebra. The method may include the step of installing a fastener on the pedicle screw such that the pedicle screw washer is located between the vertebra and the fastener.

These and other features, aspects, and advantages of the present invention will become better understood upon consideration of the following detailed description, drawings and appended claims.

### BRIEF DESCRIPTION OF THE DRAWINGS

FIG. 1 shows a perspective view of a pedicle screw washer according to one embodiment of the invention.

FIG. 2 shows a perspective view of the pedicle screw washer of FIG. 1 installed onto a vertebra.

FIG. 3 shows a side view of an orthopedic fixation system installed onto a vertebra according to another embodiment of the invention.

FIG. 4 shows a top view of the orthopedic fixation system of FIG. 3.

FIG. 5 shows a perspective view of another pedicle screw washer according to another embodiment of the invention.

FIG. 6 shows a side view of an orthopedic fixation system installed onto a vertebra according to another embodiment of the invention.

FIG. 7 shows a pedicle screw according to another embodiment of the invention.

FIG. 8 shows multiple side views of a pedicle screw washer similar to that of FIG. 1, with the top of FIG. 8 showing the pedicle screw washer before bending and the bottom of FIG. 8 showing the pedicle screw washer after bending.

Like reference numerals will be used to refer to like parts from Figure to Figure in the following description of the drawings.

### DETAILED DESCRIPTION OF THE INVENTION

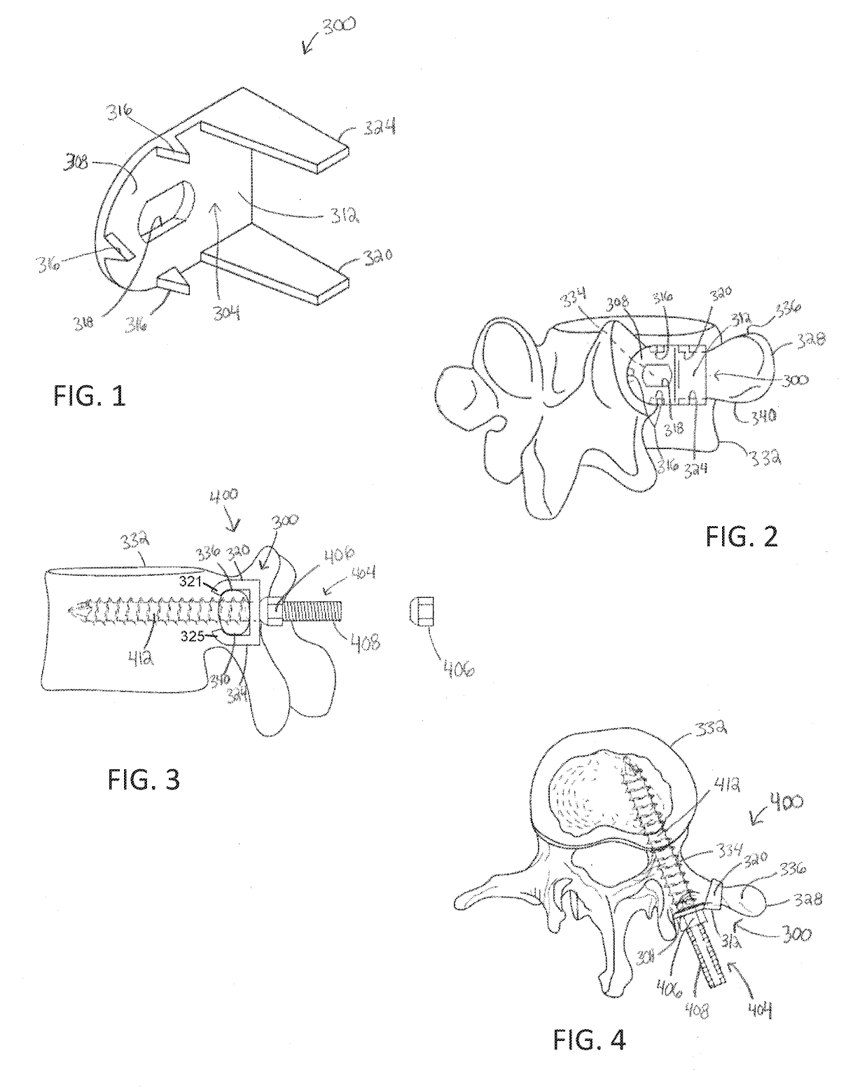

FIG. 1 shows an embodiment of a pedicle screw washer 300. The pedicle screw washer 300 is fabricated from a malleable metal, for example, a titanium alloy, stainless steel, or any other suitable malleable metal known in the art. In other embodiments, the pedicle screw washer 300 can be fabricated from a plastic material or any other suitable material known in the art. The pedicle screw washer 300 includes a body 304. The body 304 includes a first section 308 defining a substantially semi-circular edge and a second section 312 spaced from the first section 308 and defining a substantially rectangular shape. In other embodiments, the first section 308 and the second section 312 may define alternative shapes, as desired.

The first section 308 includes a plurality of bone penetrating members 316 extending from a periphery of the first section 308 of the body 304 and an aperture 318 arranged substantially centric within the first section 308. The bone penetrating members 316 define a substantially triangular shape. In other embodiments, the bone penetrating members 316 may define a substantially rounded shape, or any other shape as desired. In the illustrated embodiment, there are three bone penetrating members spaced at approximately 90 degrees increments around the periphery of the first section 308. In other embodiments, there may be more or fewer bone penetrating members 316 spaced at different increments around the periphery of the first section 308. For example, the first section may include five bone penetrating members 316 spaced at approximately 45 degree increments around the periphery of the first section 308.

In the illustrated embodiment, the aperture 318 defines a substantially rectangular shape with two opposed sides being substantially round. In other embodiments, the aperture 318 may define a different shape, as desired. For example, the aperture 318 may define a substantially round, oval, or polygonal shape.

The second section 312 includes a first securing member 320 extending from the body 304 and a second securing member 324 extending from the body 304. As shown in FIG. 1, the first securing member 320 and the second securing member 324 extend from the body 304 in a same direction as the bone penetrating members 316. The first securing member 320 and the second securing member 324 each define a substantially rectangular shape. In other embodiments, the first securing member 320 and/or the second securing member 324 may define a substantially round shape or a different shape, as desired. The first securing member 320 includes an arcuate distal end section 321 that is inwardly directed toward the second securing member 324, and the second securing member 324 includes an arcuate distal end section 325 that is inwardly directed toward the first securing member 320 (see FIG. 3).

FIG. 2 shows the pedicle screw washer 300 installed onto a transverse process 328 of a vertebra 332. As shown in FIG. 2, the pedicle screw washer 300 is installed onto the transverse process 328 such that the aperture 318 is generally aligned with a pedicle 334 of the vertebra 332 and the first securing member 320 and the second securing member 324 are generally aligned to engage the transverse process 328. The first securing member 320 is dimensioned to engage a first side 336 of the transverse process 328. The second securing member 324 is dimensioned apart from the first securing member 320 to engage a second side 340 of the transverse process 328 opposite from the first side 336.

FIG. 3 shows an application of the pedicle screw washer 300 in an orthopedic fixation system 400. The orthopedic fixation system 400 includes the pedicle screw washer 300, a pedicle screw 404, and a fastener element 406. The pedicle screw 404 includes a threaded extension 408 configured to engage the fastener element 406 and a coarsely threaded portion 412 configured to be installed into the vertebra 332.

Installation of the orthopedic fixation system 400 will be described with reference to FIG. 3 and FIG. 4. The pedicle screw 404 is installed into the vertebra 332 such that the coarsely threaded portion 412 passes through the pedicle 334 of the vertebra 332 and the threaded extension 408 protrudes from the vertebra 332. Before the pedicle screw washer 300 is installed onto the transverse process 328 of the vertebra 332, the first section 308 of the body 304 is angled with respect to the second section 312 of the body 304. This conforms the pedicle screw washer 300 to the shape of the transverse process 328, as shown in FIG. 4. The angling of the first section 308 with respect to the second section 312 may be enabled by the malleable metal in which the pedicle screw washer 300 is fabricated.

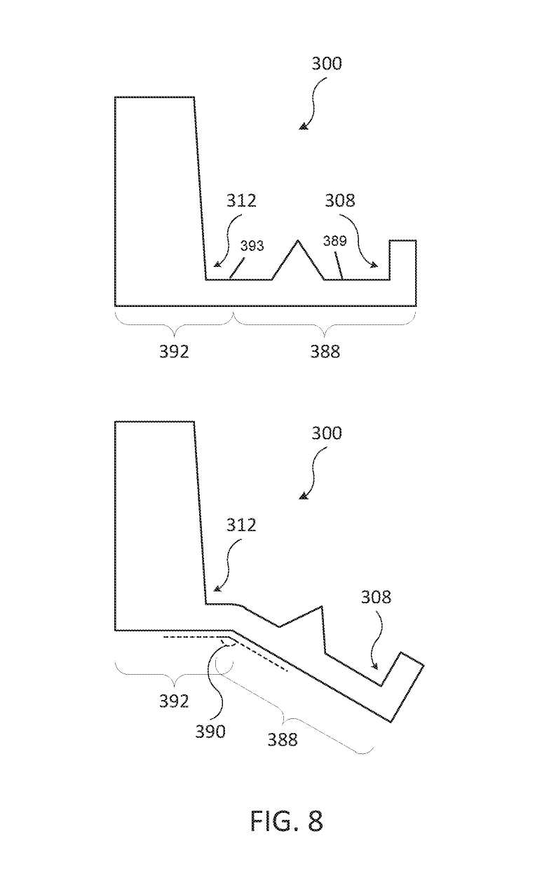

As shown in FIG. 8, the first section 308 of the pedicle screw washer 300 may have a first proximal face 388 which is located on the opposite side of a first distal face 389 from which the bone penetrating members 316 extend. Likewise, the second section 312 may have a second proximal face 392 located on the opposite side of the body 304 from a second distal face 393 from which the first and second securing members 320,324 may extend. When bent such that the first and second distal faces 389, 393 fit the bone contours along the vertebra 332 and transverse process 328, the first proximal face 388 may form an angle 390 with the second proximal face 392, as shown in FIG. 8. The angle 390 may be generally obtuse, as the vertebral arch from the pedicle 334 to the transverse process 328 generally forms an obtuse angle. However, the angle 390 may also be right or acute to best fit the particular vertebral contour.

The pedicle screw washer 300 is then arranged such that the aperture 318 is generally aligned to receive the threaded extension 408 of the pedicle screw 404 and the first securing member 320 and the second securing member 324 are generally aligned to engage the transverse process 328. The pedicle screw washer 300 is then placed into engagement with the transverse process 328. During this process, the aperture 318 receives the threaded extension 408 of the pedicle screw 404, the first securing member 320 engages the first side 336 of the transverse process 328, and the second securing member 324 engages the second side 340 of the transverse process 328. The bone penetrating members 316 are then forceably pushed into the vertebra 332. Once the bone penetrating members 316 have been pushed into the vertebra 332, the first securing member 320 is bent around the first side 336 of the transverse process 328 and the second securing member 324 is bent around the second side 340 of the transverse process 328, as shown in FIG. 3. The fastening element 406 is then installed onto the threaded extension 408 of the pedicle screw 404 thereby fastening the pedicle screw washer 300 to the vertebra 332.

With the orthopedic fixation system 400 installed onto the vertebra 332, the pedicle screw washer 300 increases the structural stability the pedicle screw 404 and aids in preventing the pedicle screw 404 from backing out of the vertebra 332. This may be accomplished by the aperture 318 engaging the threaded extension 408 of the pedicle screw 404 and the bone penetrating members 316, the first securing member 320, the second securing member 324, and the fastening element 406 structurally affixing the pedicle screw washer 300 to the vertebra 332.

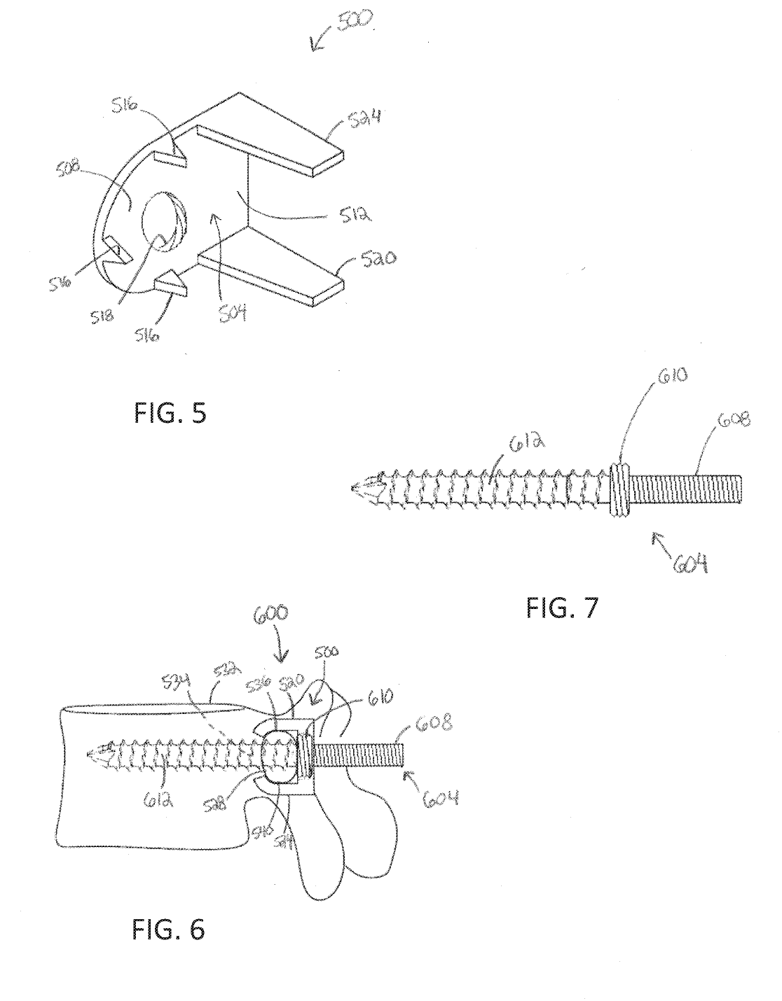

FIG. 5 shows another embodiment of a pedicle screw washer 500. The pedicle screw washer 500 may be fabricated from a malleable metal, for example, a titanium alloy, stainless steel, or any other suitable malleable metal known in the art. In other embodiments, the pedicle screw washer 500 may be fabricated from a plastic material or any other suitable material known in the art. The pedicle screw washer 500 includes a body 504. The body 504 includes a first section 508 defining a substantially semi-circular edge and a second section 512 spaced from the first section 508 and defining a substantially rectangular shape. In other embodiments, the first section 508 and the second section 512 may define alternative shapes, as desired.

The first section 508 includes a plurality of bone penetrating members 516 extending from a periphery of the first section 508 of the body 504 and an aperture 518 arranged substantially centric within the first section 508. The bone penetrating members 516 may define a substantially triangular shape. In other embodiments, the bone penetrating members 516 may define a substantially rounded shape, or any other shape as desired. In the illustrated embodiment, there are three bone penetrating members 516 spaced at approximately 90 degrees increments around the periphery of the first section 508. In other embodiments, there may be more or fewer bone penetrating members 516 spaced at different increments around the periphery of the first section 508. For example, the first section 508 may include five bone penetrating members 516 spaced at approximately 45 degree increments around the periphery of the first section 508.

In the illustrated embodiment, the aperture 518 defines a substantially round shape and includes internal threads. In other embodiments, the aperture 518 may define a different shape, as desired. For example, the aperture 518 may define a substantially rectangular, oval, or polygonal shape.

The second section 512 may include a first securing member 520 and a second securing member 524 extending from the body 504. As shown in FIG. 5, the first securing member 520 and the second securing member 524 may extend from the body 504 in a same direction as the bone penetrating members 516. The first securing member 520 and the second securing member 524 may define a substantially rectangular shape. In other embodiments, the first securing member 520 and/or the second securing member 524 may define a substantially round shape or a different shape, as desired.

As shown in FIG. 6, the pedicle screw washer 500 may be configured to be installed onto a transverse process 528 of a vertebra 532. The first securing member 520 may be dimensioned to engage a first side 536 of the transverse process 528.

The second securing member 524 may be dimensioned apart from the first securing member 520 to engage a second side 540 of the transverse process 528 opposite from the first side 536.

FIG. 6 further illustrates an application of the pedicle screw washer 500 in an orthopedic fixation system 600. The orthopedic fixation system 600 may include the pedicle screw washer 500 and a pedicle screw 604. The pedicle screw 604 may include a threaded extension 608, an externally threaded section 610 configured to engage the aperture 518 of the pedicle screw washer 500, and a coarsely threaded portion 612 configured to be installed into the vertebra 532. The threads of the coarsely threaded portion 612 may have a greater pitch than the threads of the externally threaded section 610.

Installation of the orthopedic fixation system 600 will be described with reference to FIG. 6 and FIG. 7. Before the pedicle screw washer 500 is installed onto the transverse process 528 of the vertebra 532, the first section 508 of the body 504 is angled with respect to the second section 512 of the body 504. This conforms the pedicle screw washer 500 to the shape of the transverse process 528. The angling of the first section 508 with respect to the second section 512 may be enabled by the malleable metal in which the pedicle screw washer 500 is fabricated. As described above with respect to the pedicle screw washer 300, the proximal faces 388, 392 of the first and second sections 508,512 may form an obtuse angle with respect to each other. Alternatively, the proximal faces 388, 392, opposite the bone-facing distal faces 389, 393 of the first and second sections 508,512 may be molded to form an acute or right angle with each other such that the pedicle screw washer 500 may best fit along the particular vertebral contour.

The pedicle screw washer 500 may then be arranged such that the aperture 518 is generally aligned with a pedicle 534 of the vertebra 532 and such that the first securing member 520 and the second securing member 524 are generally aligned to engage the transverse process 528. The pedicle screw washer 500 may then be placed into engagement with the transverse process 528. During this process, the first securing member 520 may engage the first side 536 of the transverse process 528 and the second securing member 524 may engage the second side 540 of the transverse process 528. The bone penetrating members 516 may then be forceably pushed into the vertebra 532. Once the bone penetrating members 316 have been pushed into the vertebra 532, the first securing member 520 may be bent around the first side 536 of the transverse process 528 and the second securing member 524 may be bent around the second side 540 of the transverse process 528, as shown in FIG. 6. The pedicle screw 604 may then be installed into the vertebra 532 such that the coarsely threaded portion 612 passes through the pedicle 534 of the vertebra 532 and the externally threaded section 610 engages the internally threaded aperture 518 of the pedicle screw washer 500. Thus, the washer 500 may be attached to the screw 604, such that the threads prevent the washer 500 from slipping off of the screw 604.

With the orthopedic fixation system 600 installed onto the vertebra 532, the pedicle screw washer 500 may increase the structural stability of the pedicle screw 604 and may aid in preventing the pedicle screw 604 from backing out of the vertebra 532. This may be accomplished by the internally threaded aperture 518 engaging the externally threaded section 610 of the pedicle screw 604 and by the bone penetrating members 516, the first securing member 520, and the second securing member 524 structurally affixing the pedicle screw washer 500 to the vertebra 532.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A pedicle screw washer comprising:
a body;
an aperture in the body for receiving a pedicle screw;
a first securing member extending from the body and dimensioned to engage a first side of a transverse process of a vertebra; and
a second securing member extending from the body and dimensioned to engage a second side of the transverse process of the vertebra.

2. The pedicle screw washer of claim 1 further comprising a bone penetrating member extending from the body.

3. The pedicle screw washer of claim 2, wherein the bone penetrating member extends from the body in a same direction as at least one of the first and second securing members.

4. The pedicle screw washer of claim 2, wherein the body includes:
a first section, including the bone penetrating member; and
a second section, including the first and second securing members.

5. The pedicle screw washer of claim 4, wherein a first proximal face of the first section of the body forms an obtuse angle with respect to a second proximal face of the second section of the body when installed on the vertebra.

6. The pedicle screw washer of claim 1, wherein the first securing member includes a first distal end section that is inwardly directed toward the second securing member, and the second securing member includes a second distal end section that is inwardly directed toward the first securing member.

7. The pedicle screw washer of claim 2, wherein the bone penetrating member extends from a periphery of the body.

8. The pedicle screw washer of claim 1, wherein a plurality of bone penetrating members extend from the body at equally spaced intervals along a periphery of the body.

9. The pedicle screw washer of claim 1, wherein the aperture includes an internal contour that mates and engages with an external contour located on a section of the pedicle screw.

10. The pedicle screw washer of claim 1, wherein the aperture includes internal detents for engaging external protrusions of the pedicle screw.

11. The pedicle screw washer of claim 1, wherein the aperture includes internal threads for engaging a threaded section of the pedicle screw.

12. An orthopedic fixation system, comprising:
a pedicle screw; and
a pedicle screw washer, the pedicle screw washer including:
a body;
an aperture for receiving the pedicle screw;
a first securing member extending from the body and dimensioned to engage a first side of a transverse process of a vertebra; and
a second securing member extending from the body and dimensioned to engage a second side of the transverse process of the vertebra.

13. The orthopedic fixation system of claim 12, wherein the pedicle screw includes an externally threaded section and a threaded extension.

14. The orthopedic fixation system of claim 13, wherein the aperture includes internal threads for engaging the externally threaded section of the pedicle screw.

15. The orthopedic fixation system of claim 13 further comprising a fastening element configured to engage the threaded extension of the pedicle screw and fasten the pedicle screw washer to the vertebra.

16. The orthopedic fixation system of claim 12, wherein a first proximal face of a first section of the body forms an obtuse angle with respect to a second proximal face of a second section of the body when installed on the vertebra.

17. The orthopedic fixation system of claim 12, wherein the first securing member includes a first distal end section that is inwardly directed toward the second securing member, and the second securing member includes a second distal end section that is inwardly directed toward the first securing member.

18. A method for securing a pedicle screw, the method comprising:
(a) placing the pedicle screw washer of claim 1 on a vertebra such that the first securing member engages a first side of a transverse process of the vertebra, and such that the second securing member engages a second side of the transverse process; and
(b) installing the pedicle screw in the vertebra and through the aperture of the pedicle screw washer.

19. The method of claim 18, wherein the aperture of the pedicle screw washer includes internal threads for engaging an externally threaded section of the pedicle screw.

20. The method of claim 18 further comprising fastening the pedicle screw washer to the vertebra by installing a fastening element on a threaded extension of the pedicle screw.

21. A method for securing a pedicle screw, the method comprising:
(a) installing the pedicle screw in a vertebra; and
(b) placing a pedicle screw washer of claim 1 on the vertebra such that the aperture in the body of the pedicle screw washer receives the pedicle screw, and the first securing member engages a first side of a transverse process of the vertebra, and the second securing member engages a second side of the transverse process.

22. The method of claim 21 further comprising bending the first and second securing members around and along the first and second sides of the transverse process of the vertebra.

23. The method of claim 21 further comprising bending the body of the pedicle screw washer to more closely follow along a contour of the vertebra.

24. The method of claim 21 further comprising forcing a bone penetrating member, extending from the body of the pedicle screw washer, into the vertebra.

25. The method of claim 21 further comprising installing a fastener on the pedicle screw such that the pedicle screw washer is located between the vertebra and the fastener.
